(19) United States
(12) Patent Application Publication
(10) Pub. No.: US 2001/0023065 A1
(43) Pub. Date: Sep. 20, 2001

(54) Method for detecting mycobacterium tuberculosis by PCR amplification of REP13E12 repeated sequence

(75) Inventors: Tae-Yoon Lee, Daegu-shi (KR); Sung-Kwang Kim, Daegu-shi (KR); Jong-Seok Lee, Daegu-shi (KR); Jai-Youl Lee, Daegu-shi (KR)

Correspondence Address: MERCHANT & GOULD, P O BOX 2903, MINNEAPOLIS, MN, 55402-0903, US

(73) Assignee: Tae-Yoon Lee

(21) Appl. No.: 09/785,904

(22) Filed: Feb. 16, 2001

(30) Foreign Application Priority Data
Feb. 19, 2000 (KR) 10-2000-7984

(51) Int. Cl. C12Q 1/68; C07H 21/04

(52) U.S. Cl. 435/6; 536/24.3

(57) ABSTRACT

A method for detecting Mycobacterium tuberculosis by the polymerase chain reaction (PCR) amplification of the REP13E12 repeated sequence, and more particularly, to a method for detecting Mycobacterium tuberculosis in clinical specimen by the PCR amplification of all or some of the REP1 3E1 2 repeated sequence is provided. Since the Mycobacterium tuberculosis detecting method by the PCR amplification for amplifying the REP13E12, which is the repeated sequence cloned from the microbial cells of Mycobacterium tuberculosis, which are separated from Korea, shows excellent sensitivity and specificity, it is possible to effectively detect Mycobacterium tuberculosis in specimen using the method.

### BACKGROUND OF THE INVENTION

1. Field of the Invention

The present invention relates to a method for detecting Mycobacterium tuberculosis by the polymerase chain reaction (PCR) amplification of the REP13E12 repeated sequence, and more particularly, to a method for specially detecting the Mycobacterium tuberculosis in clinical specimen with high sensitivity by the PCR amplification of all or some of the REP1 3E1 2 repeated sequence.

2. Description of the Related Art

Tuberculosis is a very severe infectious disease, by which ⅓ of the world population, that is, about 1,700,000,000 are infected, by which about 800,000,000 patients are newly infected every year, and from which 34% of the new patients, that is 2,700,000, die. It is estimated that the tuberculosis patients of about 700,000 exist in our country. It is reported that 140,000 patients are newly infected by tuberculosis every year and that 5,000 patients die from tuberculosis. This is a very severe public health problem.

Tuberculosis is a chronic infectious disease caused by the Mycobacterium tuberculosis. The prevalence rate of tuberculosis is increasing all over the world as well as in our country as a complication of acquired immune deficiency syndrome (AIDS). Since many of recently generated Mycobacterium tuberculosis have multiple tolerances to tuberculosis drugs, it is more difficult to cure tuberculosis.

Therefore, many-sided efforts such as development of fast diagnosis methods and new tuberculosis drugs, a molecular mechanics research, and a research on vaccine are required for elimination of tuberculosis. The most urgent one is the development of the fast diagnosis methods. Since Mycobacterium tuberculosis very slowly grow, it takes long, for example, about one through two months in order to identify Mycobacterium tuberculosis by cultivating the specimen of a patient. A method of using nucleic acid amplification such as polymerase chain reaction (PCR) amplification is used in order to cure tuberculosis at an early stage by examining Mycobacterium tuberculosis in the specimen more faster. For the PCR amplification, the base sequence of the target DNA to be amplified is necessary. The base sequence must be specific to Mycobacterium tuberculosis and must not exist in the human DNA. Many researchers have reported the base sequences specific to Mycobacterium tuberculosis.

The most representative base sequence used for the PCR amplification for detecting Mycobacterium tuberculosis is the IS6110, which is the insertion sequence (IS) specific to Mycobacterium tuberculosis. The IS6110 has the size of 1,358 bp. Both ends of the IS6110 have an inverted repeat of 30 bp. The IS6110 is inserted in zero through twenty places in the Mycobacterium tuberculosis genome. The positions, into which the IS6110 is inserted, and the number of inserted IS6110 differ in each microbial cell of Mycobacterium tuberculosis. Therefore, the IS6110 is much more useful for detecting the microbial cells of Mycobacterium tuberculosis using the PCR amplification than other base sequences specific to Mycobacterium tuberculosis. Accordingly, the IS6110 has been widely used for devising a primer for the PCR amplification.

However, it has been recently reported that Mycobacterium tuberculosis, which do not have the IS6110 or have only small number of copies, exist. In this case, another base sequence is required for detecting Mycobacterium tuberculosis by the PCR amplification. For this, it is possible to use one among the previously published base sequences specific to Mycobacterium tuberculosis. However, it is not clarified that the above base sequences specific to Mycobacterium tuberculosis with respect to the microbial cells of Mycobacterium tuberculosis divided from Korea and the specimen of Korean tuberculosis patients.

Therefore, in order to detect Mycobacterium tuberculosis using the PCR amplification in Korea, it is ideal that the microbial cells of Mycobacterium tuberculosis divided from Korea are hereditarily analyzed, that the base sequences, which commonly exist and are found only in Mycobacterium tuberculosis, are found in the DNA of the division cell of the Mycobacterium tuberculosis of Korea, and that a primer is devised on the basis of the DNA.

### SUMMARY OF THE INVENTION

The present inventor found a new repeated sequence of 453 bp from the division cell of the Mycobacterium tuberculosis of Korea and determined the base sequence (refer to Lee, T. Y. et al., Tubercle Lung Dis., 78:13-19(1997); sequence number 2). It was noted that the repeated sequence exists only in a Mycobacterium tuberculosis complex. It was also revealed later that the sequence is some of the REP13E12 (1393 bp) repeated sequence, three of which exist in the entire genome of the Mycobacterium tuberculosis standard bacilli H37Rv (refer to Cole S. T. et al., Nature, 393:537-544(1998); sequence number 1). When a new specific polymerase chain reaction (PCR) primer is devised on the basis of the sequence and the PCR amplification is performed on the clinical specimen of a tuberculosis patient, it is noted that sensitivity, which is identical or more excellent than in the PCR amplification result using the primer, which can amplify the IS6110, is shown.

Accordingly, it is an object of the present invention to provide a method for detecting Mycobacterium tuberculosis by the PCR amplification of the REP13E12 repeated sequence.

### BRIEF DESCRIPTION OF THE DRAWING(S)

The above object and advantages of the present invention will become more apparent by describing in detail a preferred embodiment thereof with reference to the attached drawings in which:

FIG. 1 is a restriction enzyme map showing the structure gene of the REP13E12;

FIG. 2 is an electrophoresis photograph showing the result of Southern blotting various acid-fast bacteria using the REP13E12 hybrid probe; and

FIG. 3 is an electrophoresis photograph showing the result of performing polymerase chain reaction (PCR) amplification on various microbial cells in order to investigate the specificity to Mycobacterium tuberculosis of the PCR amplification for REP13E12 amplification.

### DESCRIPTION OF THE PREFERRED EMBODIMENT

Hereinafter, the present invention will be described in more detail.

In the present invention, specimen (mainly sputum) is extracted from patients who are expected to be infected by tuberculosis. Mycobacterium tuberculosis are separated from the specimen, identified, and cultivated in large quantities by performing acid-fast stain. The genome DNA of each microbial cell of Mycobacterium tuberculosis is separated. The IS6110 and REP13E12 probes are fabricated, refined, and labeled for performing Southern blotting. Performing the Southern blotting checks the number of copies, in which each repeated sequence exists in the genome of the microbial cell of Mycobacterium tuberculosis. DNA of Mycobacterium tuberculosis is extracted from clinical specimen using a bead beating method. Polymerase chain reaction (PCR) amplification for IS6110 amplification (IS6110-PCR) and PCR amplification for REP13E12 amplification (REP13E12-PCR) are performed. The results are compared with the above acid-fast stain result and the cultivation result and analyzed.

As a result, at least identical or more excellent sensitivity and specificity are shown in the PCR detection of Mycobacterium tuberculosis in the specimen, which is based on the REP13E12, compared with the conventional stain and cultivation methods and the IS6110-PCR method. Therefore, the REP13E12-PCR method can be usefully and effectively used for detecting Mycobacterium tuberculosis in the specimen.

The present invention will be described more fully with reference to preferred embodiments. It will be understood by those skilled in the art that various changes in form and details may be made therein without departing from the spirit and scope of the invention as defined by the appended claims.

### EXAMPLE 1

### Processing and Primary Cultivation of Specimen

All experiments are performed using sterilized reagents and containers in a laminar flow clean bench separated from other places in a laboratory in order to minimize a false positive result due to pollution by germs.

Sputum is gathered from patients expected to be infected by tuberculosis. 4% NaOH is added to the specimen and is fully mixed with the sputum. The mixture is left for twenty minutes at a room temperature. In order to offset the buoyancy of Mycobacterium tuberculosis and concentrate Mycobacterium tuberculosis, 0.067M phosphorus buffer solution (pH 6.8) is added in 2.5 time-volume and is mixed with the resultant. The mixture is centrifuged in 3,000 g for twenty minutes. Upper aqueous phase solution is abandoned to a previously prepared abandoned solution container, which includes ethanol of 70%. Phenol red, which is a pH indicator, is dropped to a deposit. Adding 1N HCl little by little until a sample is turned yellow neutralizes the deposit. The sample is homogenized by adding 0.2% of bovine serum albumin fraction V by 1 ml. Performing the acid-fast stain on the homogenized sample and seeding in the Ogawa solid medium cultivate mycobacterium tuberculosis. Also, the remaining homogenized sample is kept at 20° C. and used for PCR analysis.

In order to primarily cultivate Mycobacterium tuberculosis in the sample, Mycobacterium tuberculosis is implanted on the medium by spreading the homogenized sample by 100 I on the surface of the 2% of Ogawa medium prepared in a tube, thus seeding Mycobacterium tuberculosis, by loosening and horizontally positioning the cover of the tube, and cultivating Mycobacterium tuberculosis in a incubator at 37° C. for twenty four hours. The cover is fastened and vertically positioned. The growth of Mycobacterium tuberculosis is observed, while cultivating Mycobacterium tuberculosis. The growth of Mycobacterium tuberculosis was observed every day until first two weeks after seeding Mycobacterium tuberculosis. After that, the growth of Mycobacterium tuberculosis was observed at three days intervals until eight weeks.

When it is suspected that the sample is polluted by common germs, which grow fast, other than Mycobacterium tuberculosis, for example, when it is confirmed that the sample is polluted by mold with the naked eye, in order to prevent equipment and other samples from being polluted, the polluted sample is separated as soon as it is found and is removed by performing steam sterilization under pressure. The incubator is sterilized by washing the incubator by 5% of phenol solution. When it is suspected that the sample is polluted, however, it is difficult to distinguish whether acid-fast bacteria or other germs pollute the sample by the naked eye, the acid-fast stain is performed. When the acid-fast bacteria pollute the sample, fluid cultivation is performed, while keeping the sample as it is, in order to reveal strain. When the sample is polluted not by the acid-fast bacteria, the sample is abandoned and sterilized like in the case, where the sample is polluted by mold.

### EXAMPLE 2

### Acid-Fast Stain

After common germs are removed from specimen by 2% of NaOH, to which 0.5% of N-acetyl-L-cysteine is added, the resultant is centrifuged in 4,000 g for five minutes, thus concentrating Mycobacterium tuberculosis. The resultant is acid-fast stained and is observed by a microscope. The acid-fast stain is performed by the Ziehl-Neelsen method. The stain result is shows by a center for disease control (CDC) index. After a culdoscope is performed more than 100 fields, it is determined that it is negative (more than three times at a long axis and nine times at a short axis).

*CDC index

| | |
|---|---|
| 1 through 2/300 vision: (±) | 1 through 9/100 visions: (1+) |
| 1 through 9/10 visions: (2+) | 1 through 9/visions: (3+) |

>9/visions: (4+)

### EXAMPLE 3

### Cultivation of Mycobacterium Tuberculosis

First, it is confirmed that the germs grown in the solid medium of the embodiment 1 are acid-fast germs by the acid-fast stain. Since an enough amount of germs must be secured in order to keep isolates and perform experiments, the germs are obtained from the Ogawa solid medium of the embodiment 1 and seeded in the Middlebrook 7H9 liquid medium (Difco of USA) including the Albumin fraction V:bovine, Dextrose and Catalase; Difco of USA (ADC). The liquid medium containing the germs is lightly shaken at about 100 rpm and is cultivated until the amount of germs becomes enough at 35° C. through 37° C. for more than four weeks. At this time, the Middlebrook 7H9 liquid medium is manufactured by dissolving 4.7 g of Middlebrook 7H9 medium powder per 900 ml of distilled water, performing steam sterilization under pressure, adding 2 ml of glycerol, which is steam sterilized under pressure, mixing the glycerol with the resultant, and adding 10 ml of Middlebrook ADC to the mixture.

After collecting the germs by centrifuging the cultivated germs at 3,000 ×g for twenty minutes, some of them are kept at −70° C. after adding Brucella broth and 15% of glycerol and the others are kept at −20° C. and are used for various experiments.

### EXAMPLE 4

### Separation of Mycobacterium Tuberculosis Genome DNA for Southern Blotting

After the Mycobacterium tuberculosis cultivated in large quantities in the example 3 are collected and left at 75° C. for twenty minutes, thus weakening poison so that the collected Mycobacterium tuberculosis is used to split DNA. Mycobacterium tuberculosis is used for separating DNA. After Mycobacterium tuberculosis are frozen at −70° C. and melt, lysozyme is added to Mycobacterium tuberculosis at the density of 2 mg/ml and the resultant is left at 37° C. for one hour. After adding 1% of SDS and 1 mg/ml of proteolytic ezenzyme K (proteinase K), the resultant is left at 55° C. for 48 hours. The resultant is cleaned at 55° C., for 30 minutes, and two times by TE buffer solution containing 0.04 mg/ml of phenylmethylsulfonylfluoride, which is an inhibitor of the proteolytic ezenzyme K. After that, after the same amount of chloroform-isoamylalcohol mixture solution (chloroform:isoamylalcohol=24:1 (v/v)) is added and mixed with the resultant, the mixture is centrifuged, upper aqueous phase solution is obtained, and isopropanol of 0.6 time-volume. Accordingly, the genome DNA is sedimented.

### EXAMPLE 5

### Mycobacterium Tuberculosis DNA Separation for PCR Analysis

In order to separate DNA from clinical specimen, which is the subject of detection of Mycobacterium tuberculosis by the PCR application, simple processes with no factor of preventing the PCR reaction are necessary. DNA is separated by the bead-beating method of physically breaking cells and extracting DNA by phenol/chloroform/isoamylalcohol mixture solution (phenol:chloroform:isoamylalcohol =25:24:1(v:v), hereinafter PCI). First, after minimizing the amount of contained mucin component by dissolving the clinical specimen such as sputum by 4M NaOH, the resultant is centrifuged. Accordingly, Mycobacteria, which might be contained inside the resultant, are collected. After removing the upper aqueous phase solution, 100 I of zirconium bead (Biospec Products of USA) having a diameter of 0.1 mm, which is contained in the distilled water, is added. After adding 100 I of distilled water and 100 I of PCI, the resultant is shaken for three minutes using the mini-bead beater (the Biospec Products of USA) and centrifuged at 12,000 rpm. Accordingly, an upper aqueous phase is separated from the resultant. After adding 5M NaCl of ⅕ volume and 10% of cetylmethylammonium bromide-0.7M NaCl of ⅛ volume, the DNA is left at 65° C. for 10 minutes. By adding isopropanol of 0.6 times volume after processing with the same amount of PCI and removing the aqueous phase, the DNA is deposited. The deposited DNA is dissolved in sterile distilled water of a proper volume and preserved at −(minus) 20° C. so that the DNA is used in PCR.

### EXAMPLE 6

### Southern Blotting

### Example 6-1

### Preparing Southern Blotting Probe DNA by Using IS6110

245 bp DNA which a part of Mycobacterium tuberculosis specific base sequence IS6110 is amplified by PCR and used as a probe. Primer base sequence and PCR reaction for the amplification thereof is identified with the method which will be described in example 7. The amplified fragment is split by an 8% polyacrylamide gel electrophoresis, DNA with size of 245 bp is left by crush and soak method at 37C as it is, then DNA is used as probe DNA by eluation, purification by phenol processing, and precipitation by ethanol.

### Example 6-2

### Preparing Southern Blotting Probe DNA by Using REP13E12

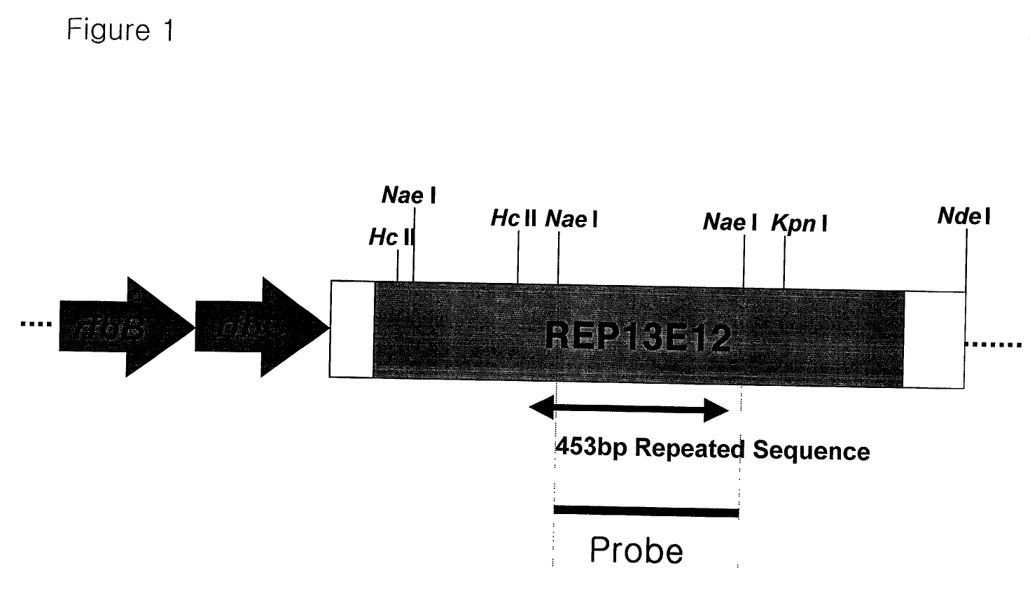

A fragment of 430 bp is split and purified by the same method used in the example 6-1 after splitting the fracturing plasmid pIS116(refer to Lee, T. Y. et al., Tubercle Lung Dis., 78:13-19 (1997)) comprising 453 bp repeated sequence which belongs to REP13E12 with Nae . FIG. 1 is a restriction map showing a structural gene of REP13E12. In FIG. 1, rfbB and rfbC show a part of rhamnose biosynthesis gene, probe shows a probe DNA for PCR amplified southern blotting as described above.

### Example 6-3

### Probe Marker

The DNA for a probe split and purified in the example 6-3 is marked by using DIG DNA marker and detection kit(Boehringer Mannheim, Germany). That is, after denaturating the purified DNA as single strand by letting the purified probe DNA 15 I in boiled water for 10 minutes and mixing the DNA with dNTP of 2 15 I in the ice, Klenow fragment of 1 15 1 is added to the mixed DNA. Being reacted for above 16 hours at 37° C. marks the DNA. After this, EDTA(0.2M, pH 8.0) of 2 15 I and 4M LiCl of 2.5 15 I are added to the DNA and the DNA is used after deposition by ethanol.

Example 6-4

### Southern Hybridization

Mycobacterium tuberculosis genome DNA split in the example 4 is split with Pvull, and an electrophoresis is executed in two 0.8% agarose gels. The electrophoresis is executed in tris-acetate-EDTA buffer solution for 8-10 hours at constant voltage of 40V. Being put in ethidium bromide solution for proper time stains the gel, then the southern blotting is executed after shooting photograph by putting a ruler as follows:

The gel is left in a denaturation buffer solution (0.5M Na OH, 1.5M Na Cl) for 15 minutes, then is put in neutralization buffer solution (0.5M Tris-HCl containing 3M NaCl, pH 7.5) for above 30 minutes. The DNA in the gel is dried after being transferred to a Nylon film filter (Amersham International, UK), and fixed to a film by being left for 2 hours at 80° C. After this, the gel is prehybridized in hybridization solution (5×SSC, 50% Formamide, 0.1% N-lauroylsarcosine) and 0.02% SDS, 2% blocking reagent and the solution is wasted. Then, replacing the prehybridization solution with a solution that the probe (manufactured in the example 6-3) denaturated to single-strand by boiling is added the hybridization solution, the DNA is reacted for more than one hour. Next, being cleaned with cleaning buffer solution of two-times concentration (2×SSC, 0.1% SDS) at normal temperature by two times, cleaned with cleaning buffer solution of 0.5-times concentration (0.5×SSC, 0.1% SDS) at 55° C. by two times, and adding a blocking solution (maleic acid buffer solution containing 1% blocking reagent) at a degree that the film is soaked, the DNA is left for one hour. After reacting the DNA in the Diluted blocking solution with anti-DIG alkaline phosphatase by 5000-times for 30 minutes, the color development of the DNA is performed at a dark place of normal temperature by removing the rest of the non-coupled anti-DIG alkaline phosphatase by washing the DNA with a washing buffer solution for detection (1.5 ml per buffer solution of 500 ml maleic acid) two times for 15 minutes and adding NBT (4-nitro blue tetrazolium chloride) of 45 μl and X-phosphatase of 35 μl to the buffer solution of 10 ml for detection (0.1M Tris-HCL, pH 9.5 containing 0.1M NaCl, 0.05M MgCl₂).

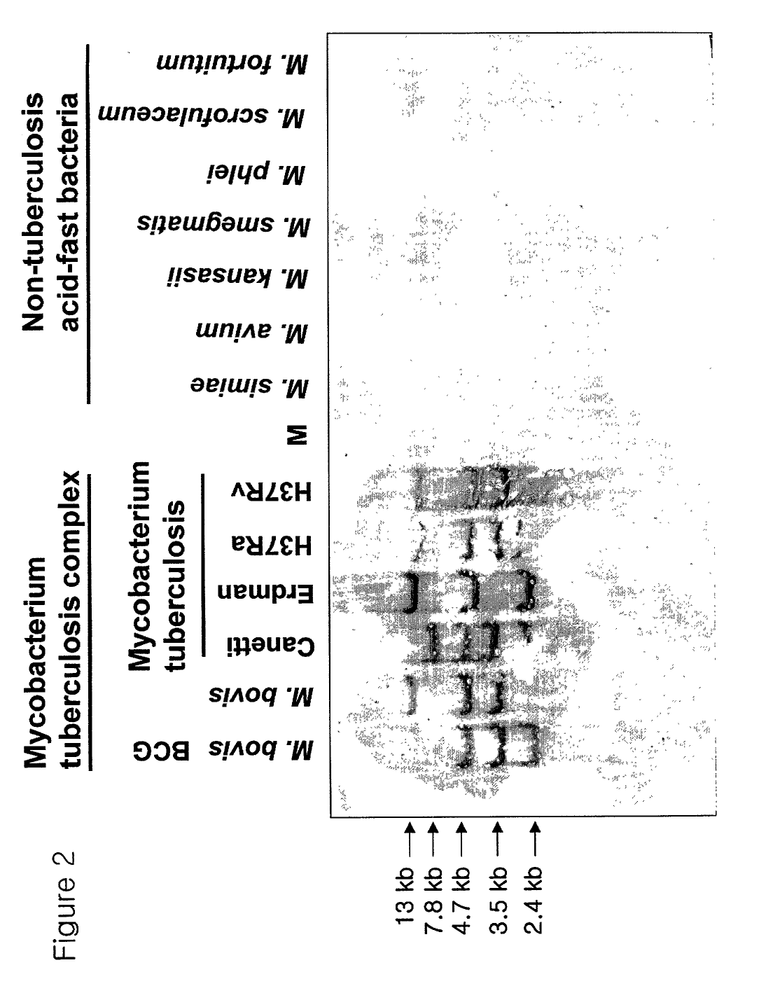

FIG. 2 is an electrophoresis picture showing the result of the southern blotting for various acid fast organism by using REP12E12 prehybridization probe manufactured in the example 6-3. As shown in FIG. 2, it can be understood that REP13E12 probe is especially presented in bacterial strain belong to Mycobacterium tuberculosis complex, and 3-4 copies are presented in accordance with the bacterial strain.

### EXAMPLE 7

### PCR

A primer based on 454 bp repeated sequence cloned from Mycobacterium tuberculosis of Korea (Lee, T. Y. et al., tubercle Lung Dis., 78:13-19 (1997): this belongs to a part of REP13E12 repeated sequence (sequence No.1) of Mycobacterium reference strain, H37Rv) and Mycobacterium specific primer INS-1/INS-2 (sequence Nos. 5 and 6) based on the previous used IS6110 are used as Mycobacterium specific PCR.

PCR reacting mixed solution contains 10 mM Tris-HCl (pH 8.8), 50 mM KCl, 1.5 mM MgCl₂, 0.1% Triton X-100, the above primers of 0.5 mM, Tag polymerase of dNTPs of 200 mM and 2.5 units (DyNaZyme, Finland). The PCR is performed after adding template DNA of 1 ng to the PCR reacting mixed solution (total volume: 50μl). To prevent vaporization of moisture during the reaction, mineral oil is dropped to the reaction mixed solution one by one. Circulation of temperature is performed one time for 10 minutes at 97° C., for 2 minutes at 60° C., and for 3 minutes at 72° C.; 30 times for 1 minute at 96° C., for 2 minutes at 60° C., and for 3 minutes at 72° C.; and 1 time for minute at 60° C. and for 10 minutes at 72° C.

dUTP is used to dNTP in stead of dTTP, The PCR is performed after destroying the amplicon by leaving the OCR reacting mixed solution for 10 minutes at 50° C. by adding 1 (one) unit of uracil DNA N-glycosilase.

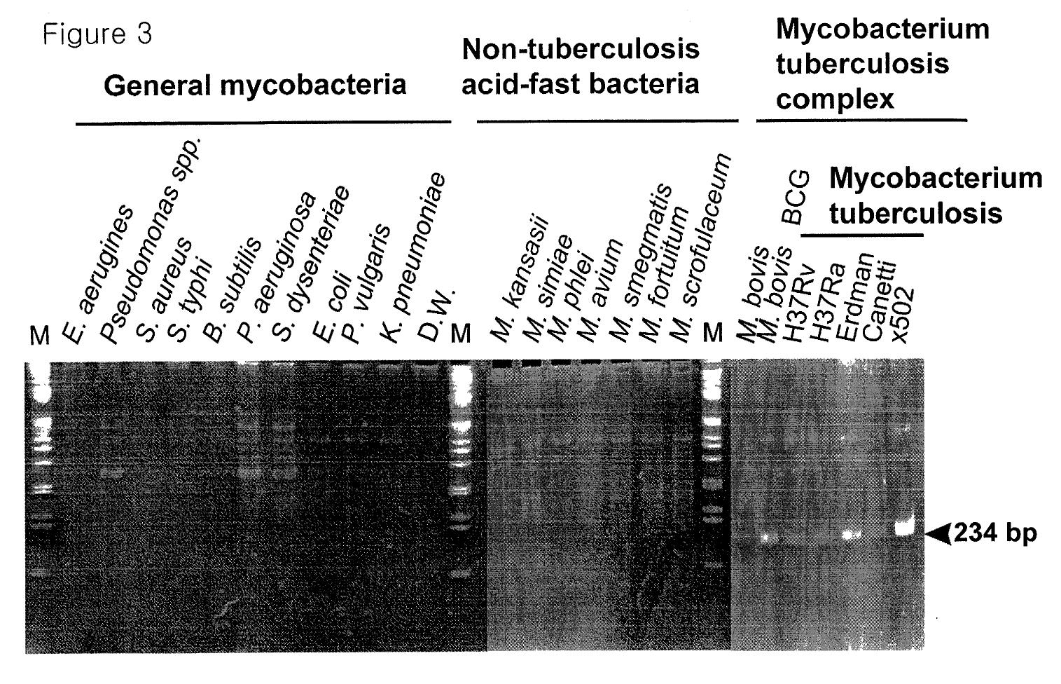

FIG. 3 is an electrophoresis picture showing the result of PCR with respect to various bacterial strains for investigation of the particularity of Mycobacterium of REP13E12-PCR. In FIG. 3, x502 is represented Mycobacterium separated from sputum of Korean Mycobacterium patient. As shown in FIG. 3, it can be understood that an expected band, 234 bp band is observed only the bacterial strains belong to Mycobacterium complex. Accordingly, it is confirmed that PCR amplifying REP13E12 is specific to Mycobacterium complex.

According to the above examples 1-7, acid-fast stain, culture, and investigation of the presence of Mycobacterium with respect to medical specimen (sputum) collected from Korean Mycobacterium patients, and comparing these are performed (table 1).

TABLE 1
Result of detecting Mycobacterium in the specimen by acid-fast
stain, culture, IS6110-PCR and REP13E12-PCR.

| | Bacterial | No. of | Result of | Result of | Result of PCR | |
|---|---|---|---|---|---|---|
| Group | strain | Specimen | Stain | Culture | IS6110 | REP13E12 |
| I | Tb | 72 | + | + | + | + |
| | Tb | 10 | − | + | + | + |
| II | Tb | 2 | + | − | + | + |
| | Tb | 1 | − | − | + | + |
| III | Tb^{a} | 3 | + | − | + | − |
| | Tb^{a} | 5 | + | − | − | + |
| IV | MOTT | 3 | + | + | − | − |
| | MOTT | 1 | − | + | − | − |
| V | FP | 1 | − | − | + | − |
| VI | MOTT^{b} | 14 | + | − | − | − |
| VII | None | 156 | − | − | − | − |

Tb: M. tuberculosis
Tb^{a}: There is probability that M. tuberculosis exists.
MOTT: Mycobacteria other than tubercle bacilli
MOTT^{b}: There is probability that Mycobacteria other than tubercle bacilli exists.
FP: Positive false as a result of the IS6110 PCR is shown.

As shown in Table 1, Mycobacterium tuberculosis are cultivated in group I regardless of the result of stain and the DNA of the Mycobacterium tuberculosis is detected in the two PCRs, that is, the IS6110-PCR and the REP13E12-PCR. Total 82 specimens belong to the group I. In the group II, Mycobacterium tuberculosis are not cultivated regardless of the result of the stain. The DNA of Mycobacterium tuberculosis is detected in the IS6110-PCR and the REP13E12-PCR. Total 3 specimens belong to the group II. In group III, the DNA of Mycobacterium tuberculosis is detected in either the IS6110-PCR or the REP13E12-PCR and Mycobacterium tuberculosis are not cultivated. However, the acid-fast germs are detected as the result of the stain. It is strongly estimated that Mycobacterium tuberculosis exist in the group III due to the specificity of Mycobacterium tuberculosis of the repeated sequence used in the present experiment. Total 8 specimens belong to the group III. It is determined that Mycobacterium tuberculosis exist in the specimen in the above 3 groups, which are considered in the subsequent result analysis.

In group IV, the germs are cultivated regardless of the result of the stain. However, the colony of the cultivated germs assumes an aspect different from Mycobacterium tuberculosis and is negative in the 2 PCRs. The 4 microbial cells are assumed to be MOTTs. In group V, the germs are negative to other experiments excluding the IS6110-PCR, to which the germs are positive. Total 1 specimen belongs to the group IV. Positive false as a result of the PCR amplification is assumed to shown. A case, where the germs are negative to other experiments excluding the REP13E12-PCR, is not observed. In group VI, the germs are negative to all experiments, however, it is determined that the acid-fast germs exist in the result of the stain. Total 14 specimens belong to the group VI. In group VII, it is determined that Mycobacterium tuberculosis or the acid-fast germs do not exist in all examination methods. Total 156 specimens beong to the group VII.

Sensitivity, specificity, a positive predictive value (PPV), and a negative predictive value (NPV) in the detection of Mycobacterium tuberculosis of the respective examination methods are analyzed on the basis of the examination results and are shown in Table 2. At this time, the number of specimens, which are expected to contain Mycobacterium tuberculosis, is totally 93, that is, from the group I through the group III as shown in Table 1. These are analyzed as golden positive standards. The number of specimens, which are golden negative standards, is totally 175 from the group IV through the group VII.

TABLE 2
analysis of the sensitivity and the specificity of the result of the PCR
amplification compared with those of the results of the cultivation and the stain

| kind of experiment | Number of specimen | | Sensitivity | specificity | PPV | NPV |
|---|---|---|---|---|---|---|
| and result thereof | Positive | Negative | (%) | (%) | (%)^{a} | (%)^{b} |
| Stain | | | | | | |
| Positive | 82 | 17 | 88.2 | 90.3 | 82.8 | 93.5 |
| Negative | 11 | 158 | | | | |
| Culture | | | | | | |
| Positive | 82 | 4 | 88.2 | 97.7 | 95.3 | 94.0 |
| Negative | 11 | 171 | | | | |
| IS6110-PCR | | | | | | |
| Positive | 88 | 1 | 94.6 | 99.4 | 98.9 | 97.2 |
| Negative | 5 | 174 | | | | |
| REP13E12-PCR | | | | | | |
| Positive | 90 | 0 | 96.8 | 100 | 100 | 98.3 |
| Negative | 3 | 175 | | | | |

As shown in Table 2, the PCR method is much excellent in respect of sensitivity, which was noted from the results of conventional researches. when the two PCR methods are compared with each other, the REP13E12-PCR seems to be slightly more excellent in respect of sensitivity. However, no statistical difference exists. also, in Table 2, the sensitivity is the same in the stain result and the cultivation result. This is because the stain result is positive although the acid-fast germs excluding Mycobacterium tuberculosis exist. The cases, which significantly contribute to this, are the group VI.

The cultivation method is more excellent than the stain method in respect of specificity as expected. That the degree of specificity is a little lower in the cultivation method, which was reported to be more excellent than the PCR method in respect of the specificity, is because the result of four MOTTs (group IV) is included. if anyone skilled in the art cultivates the germs, the specificity of the cultivation method is 100%. in the case of the PCR method, the false positivity is pointed as a large problem. However, in the present experiment, the false positivity is shown only in the IS6110 -PCR and very excellent specificity is shown in the two PCR methods.

PPV is a probability, in which the positivity is proved to be true when it is determined to be positive by a certain examination method. NPV is a probability, in which the negativity is proved to be true when it is determined to be negative by a certain examination method. the PCR method is more excellent than the cultivation method in respect of the PPV and THE npv. The REP13E12-PCR method is slightly more excellent than the IS6110 -PCR method in respect of the PPV and the NPV.

Therefore, the REP13E12-PCR method, whose effectiveness is first examined in the present invention, is at least equal to or more excellent to the IS6110 -PCR method in respect to the sensitivity, the specificity, the PPV, and the NPV. The REP13E12-PCR method can be usefully used for detecting Mycobacteria tuberculosis in the specimen.

What is claimed is:

1. A method for detecting Mycobacterium tuberculosis using polymerase chain reaction (PCR) amplification with respect to all or some of the REP13E12 repeated sequence, which exists only in Mycobacterium tuberculosis complex.

2. The method of claim 1, wherein the REP13E12 repeated sequence is consist of the base sequence corresponding to the sequence number 1.

3. The method of claim 1, wherein the PCR amplification with respect to some of the REP13E12 is performed by a pair of primers devised on the basis of the sequence number 2.

4. The method of claim 1, wherein Mycobacterium tuberculosis is the tuberculosis microbial cells separated from Korea or from the specimen of Korean tuberculosis patients.

5. The method of claim 3, wherein the pair of primers are the sequence numbers 3 and 4.


SEQUENCE LISTING (6 sequences)

SEQ ID NO: 1 - DNA, 1393 bp, Mycobacterium tuberculosis
tgggttcgggtagccgcgaacggattgtcgaggtctttgatgcgctggatgccgagctggaccgcttggacgaggtgtcttttgaggtgttgaccaccccagaacggctgcggtctctggaacgtctggaatgcttggtgcgccggctaccggcggtgggtcacgcgttgatcaaccaacttgacgcccaagccagcgaggaagaactgggcggcacgctgtgctgcgcgctggccaaccggttacgcatcaccaagcccgacgccgcccggcgcatcgccgacgccgccgatctcggacctcgtcgagcactcaccggtgaaccgctagccccacagttgaccgccaccgccaccgcccaacgccagggcctgatcggcgaggcgcacgtcaaagtgattcgcgccctttttcgcccacctgcccgccgcggtggatgtgtccacccgccaggccgccgaagccgacctggccggcaaagccgctcaatatcgtcccgacgagctggcccgctacgcccagcgggtcatggactggctacaccccgacggcgacctcaccgacaccgaacgcgcccgcaaacgcggcatcaccctgagcaaccagcaatacgacggcatgtcacggctaagtggctacctgaccccccaagcgcgggccacctttgaagccgtgctagccaaactggccgcccccggcgcgaccaaccccgacgaccacaccccggtcatcgacaccacccccgatgcggccgccatcgaccgcgacacccgcagccaagcccaacgcaaccacgacgggctgctggccgggctgcgcgcgctgatcgcctccgggaaactgggccaacacaacggtcttcccgtctcgatcgtggtcaccaccaccctgaccgacctgcaaaccggcgccggcaagggcttcaccggcggcggcaccctgctacccatggccgatgtgatccgcatgaccagccacgcccaccactactcccccgcaagcgggaggtacccccaggcgatcttcgaccacggcacacccctggcgctgtatcacaccaaacgcctagcctccccggcccagcggatcatgctgttcgccaacgaccgcggctgcaccaaacccggctgtgacgcaccggcctaccacagccaagcccaccacgtcaccgcctggaccagcaccggacgcaccgacatcaccgagctgaccctggcctgcggccccgacaaccgactcgccgaaaaaggctggaccacccacaacaacacccacggccacaccgaatggctaccaccaccccacctcgaccacggccaaccccgcaccaacaccttccaccaccccgaacgattcctccacaaccaagacgacgacgacaaacccgattgac

SEQ ID NO: 2 - DNA, 453 bp, Mycobacterium tuberculosis
gatcggcgaggcgcacatcaaagtgattcgcgccctttttcgcccacctgcccgccgcggtggatgtgtccacccgccaggccgccgaagccgacctgccggcaaaggcctcaatatcgtcccgacgagctggcccgctacgcccagcgggtcatggactggctacaccccgacggcgacctcaccgacaccgaacgcgcccgcaaacgcgcatcaccctgagcaaccagcaatacgacggcatgtcacggctaagtggctacctgaccccccaagtcgcgggccacctttgaagccgtgctagccaaactggccgcccccggcgcgaccaaccccgacgaccacaccccggtcatcgacaccacccccgatgcggccgccatcgaccgcgacacccgcagccaagcccaacgcaaccacgacgggctgctggccgggctgcgcgcgctgatc

SEQ ID NO: 3 - DNA, 18 bp, Artificial Sequence
  Single stranded oligonucleotide primer
acatcaaagtgattcgcg

SEQ ID NO: 4 - DNA, 18 bp, Artificial Sequence
  Single stranded oligonucleotide primer
catgccgtcgtattgctg

SEQ ID NO: 5 - DNA, 21 bp, Artificial Sequence
  Single stranded oligonucleotide primer
cctgcgagcgtaggcgtcggt

SEQ ID NO: 6 - DNA, 20 bp, Artificial Sequence
  Single stranded oligonucleotide primer
ctcgtccagcgccgcttcgg